(19) United States
(12) Patent Application Publication
(10) Pub. No.: US 2006/0116630 A1
(43) Pub. Date: Jun. 1, 2006

(54) Method & apparatus for pressurizing a body cavity for diagnostic and rehabilitative purposes

(76) Inventor: Antoine Garabet, Glendora, CA (US)

Correspondence Address: JAMES E. BRUNTON, ESQ., P. O. BOX 29000, GLENDALE, CA, 91209, US

(21) Appl. No.: 11/000,839

(22) Filed: Nov. 30, 2004

(51) Int. Cl. A61M 31/00

(52) U.S. Cl. 604/65

(57) ABSTRACT

A method and apparatus for both the diagnostic measurement and therapeutic treatment of a body cavity. According to one form of the method of the invention, a fluid (liquid or gas) under pressure is introduced into a selected body cavity while monitoring the pressure or flow of the fluid (liquid or gas) into the cavity. Following pressurization of the body cavity, fluid (liquid or gas) inflow and outflow data is collected and analyzed. The data collected is used to draw various conclusions about the biomechanical properties of the body cavity and the organ in which it is present, and also to draw conclusions about presence or absence of disease as well as the character of disease. According to another method of the invention a fluid (liquid or gas) is controllably infused into the body cavity to controllably expand the body cavity for purposes of therapeutic treatment.

### BACKGROUND OF THE INVENTION

1. Field of the Invention

The present invention relates generally to diagnostic measurements and therapeutic treatment of body cavities. More particularly, one form of the invention concerns a method and apparatus for making dynamic biomechanical measurements of a body cavity. More specifically, the method involves the use of pressure and flow of fluid (liquid or gas) in and out of the body cavity to characterize the elastic properties of the cavity wall and to draw conclusions as to the presence or absence of disease in the organ that contains the cavity. Another form of the invention concerns controlled pressurization of the body cavity for therapeutic purposes, which includes both pain/discomfort reduction and motility rehabilitation.

2. Discussion of the Prior Art

In the past pressurized inflation of body organs and body cavities has been used in a variety of fashions. Some of these uses include air inflation of a body cavity for purposes of visualization (colonoscopy, laperoscopy) and others to deliver vital oxygen into the lungs (artificial ventilation), and intrauterine insufflation to check for potency during an ablative procedure. The use of pressure to extrapolate mechanical properties of tissue or body cavities has also been used to a limited extent in pulmonary function testing.

Other existing technology uses a balloon inserted in the rectum to measure threshold for sensitivity or pain in individuals with Irritable Bowel Syndrome. The machine is called a ‘Barostat’ (Brouin M., Gastroenterology, 10 Jun. 2000: 122(7): 1771-7). The Barostat can also be used to measure stress/strain relationship of the wall of the rectum (Whitehead W E, Dig. Dis. Sci, 01 Feb. 1997: 42(2): 223-41; Disrutti E., Gastroenterology, 01 May 1999: 116(5): 1035-42). The current invention measures biomechanical properties without the use of a balloon. The Barostat is not used for therapeutic applications like the current invention.

Biologic tissue is known to be viscoelastic in nature, a property that is known to be altered by disease state.

In Irritable Bowel Syndrome, the colon loses its ability to coordinate movement due to increase, decrease, or alteration in its muscular tone. However, as will become apparent from the description which follows, these spastic or flaccid tendencies are measured and treated by the method of the present invention.

### SUMMARY OF THE INVENTION

The method and apparatus described in the present invention involves the controlled infusion of a fluid (gas or liquid) into a body cavity both for therapeutic purposes as well as for the purpose of ascertaining the dynamic biomechanical properties of the organ in which the cavity is contained. The methods of the invention are diverse and cover many aspects of medicine. In particular, the diagnostic methods of the invention are concerned with two types of biomechanical measurements, namely measurements during pressurization (load) and during depressurization (unload). Loading occurs when the fluid (liquid or gas) is actively infused into the body cavity, and unloading occurs when the fluid (liquid or gas) is passively allowed to be expelled from the cavity by the organ's own biomechanical rebound.

In one form of the method of the invention, a flexible tube is used to connect the body cavity to the apparatus of the invention and this novel method includes the following features:
(a) a pressurizing system capable of producing fluid (liquid or gas) which is calibrated in a manner that is to accurately measure pressure and/or flow during the load phase;
(b) a system that is capable of controllably delivering the fluid (liquid or gas) in a safe manner;
(c) a system that includes tubing that permits the efficient delivery and infusion of the fluid (liquid or gas) into the body cavity;
(d) a system that includes means for accurately monitoring pressure and/or flow characteristics during the inflow or load phase;
(e) a system that includes tubing that enables the fluid (liquid or gas) to be expelled from the body cavity and delivered into a discharge bag;
(f) a system that enables monitoring of pressure and/or flow during the outflow or unload phase;
(g) a system that enables data collection for purposes of interpretation during both the inflow and outflow phases; and
(h) a system that provides interactive features which permit the user to adjust the parameters of pressure and/or flow, based on the nature of the body cavity being investigated, including:
   choosing the initiation pressure during the inflow phase;
   choosing the pressure level at which the loading phase ends and the unloading phase begins; that is, during the inflow/outflow transition;
   choosing the pressure level at which the unloading phase is terminated;
   choosing the maximum pressure allowable within the body cavity during the pressurizing phase;
   choosing the maximum volume allowable for infusion in the body cavity; and
   a system which allows for automatic cessation of pressurization, along with the opening of a safety valve if the preset maximum pressure is exceeded.

Once the user of the apparatus initiates a specific study, the study will proceed from inflow to outflow without interruption so long as the pressure within the body cavity remains within predetermined levels. In this regard, the system is designed to permit the user to reset the system and repeat the study under the same or different parameters.

In the one form of the diagnostic method of the invention, the apparatus allows for data collection and storage capabilities so that the pressure and flow data can later be used for purposes of interpretation as to the dynamic loading/unloading biomechanical properties of the cavity under investigation. Ultimately, the same data can also be used for therapeutic planning.

The therapeutic features of the invention allow for the following:
a system to pressurize a cavity (colon) in a certain therapeutic fashion to reduce pain or abnormal perceptions/sensations.
a system to allow the tissue (musculature of the colon) to exert effort to expel fluid (liquid or gas) at a certain controlled predetermined fashion.
a system to tailor the inflation/deflation parameters based on the disease process of the organ in question.
a system to manipulate the parameters of therapeutic inflation/deflation based on biomechanical feedback system and on the pain/discomfort reduction effectiveness, or other subjective perceptions of relief expressed by the patient.

The apparatus of the invention can also be used for motility rehabilitative purposes. More particularly, the apparatus can be used to train body cavities with motility dysfunction to function more normally. This can be achieved with repetitive inflation and deflation of the body cavity. With repeated training of the body cavity using the device, the organ can be rehabilitated in a manner to achieve for long-term, sustainable normal functional motility.

When used for rehabilitation purposes the apparatus:
(a) can be used to pressurize a selected body cavity in a repetitive fashion as predetermined by the user;
(b) can be used to determine the strengths and weaknesses of the body cavity, hence allowing the user to design a treatment plan;
(c) can be used to carry out procedures that are customized for the unique biomechanical features of a body cavity;
(d) can be used to restrain and restrict or even stimulate the mechanical response of a body cavity to a finite and predetermined range;
(e) can be used to cause the body cavity to be rehabilitated and “rewired” neurologically for proper motility and biomechanical functioning; and
(f) can be customized for each individual patient using modes and settings of the system. These variables include: flow rate, pressure, pressure rate change, interval between cycles, amplitude of cycle, variations between cycles, resistance of outflow, and number of cycles. Variations can also be made within same treatment mode (intratreatment variation), variation from one treatment to the next (intertreatment variations) and interactive variations (programmed).

### BRIEF DESCRIPTION OF THE DRAWINGS

FIG. 1 is a generally perspective view of one form of the apparatus of the invention for making biomechanical measurements of a body cavity.

FIG. 2 is a generally diagrammatic view illustrating the various components that make up apparatus of the invention shown in FIG. 1.

FIG. 3 is a generally diagrammatic view illustrating the operational interrelationship among the various components of one form of the apparatus of the invention.

FIG. 4 is a generally diagrammatic view illustrating the sequence of operation of one form of the priming method of the invention.

FIG. 5 is a generally diagrammatic view further illustrating the sequence of operation of one form of the apparatus of the invention.

FIG. 6 is a generally diagrammatic view illustrating the automatic termination feature of the invention.

FIG. 7 is a graphical representation illustrating operational pressures as a function of time during the performance of an alternate, diagnostic form of the method of the invention.

FIG. 8 is a generally diagrammatic view further illustrating the sequence of operation of an alternative rehabilitation form of the apparatus of the invention.

FIG. 9 is a graphical representation illustrating pressure as a function of time during the rehabilitation method of the invention.

### DESCRIPTION OF THE INVENTION

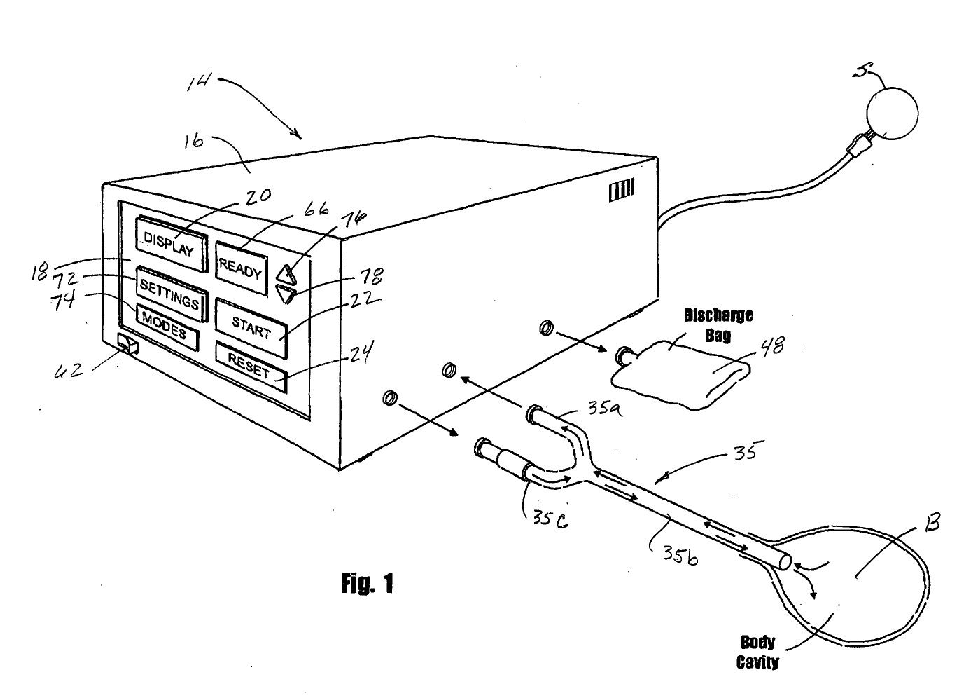

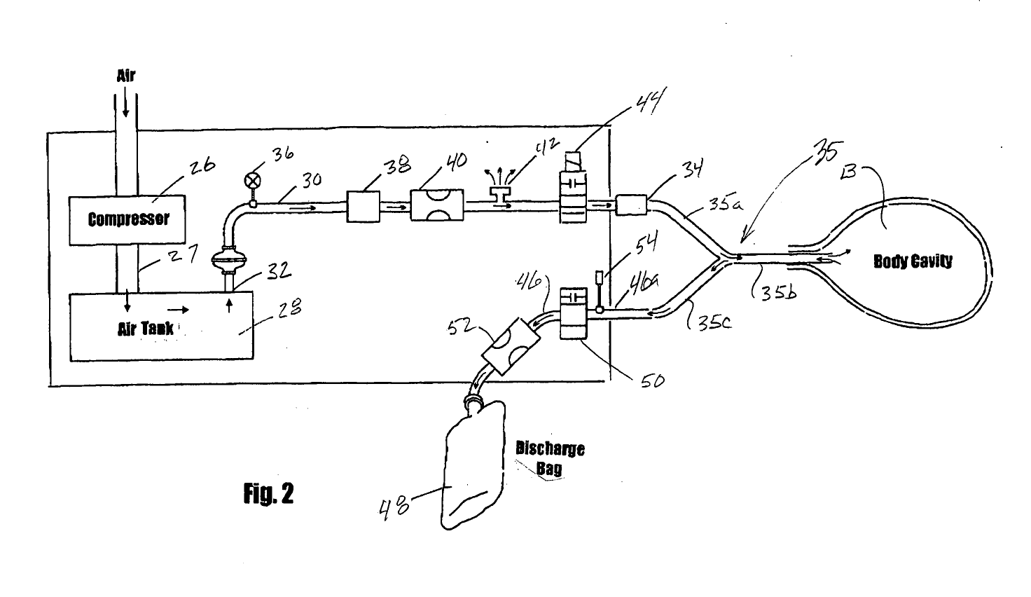

Referring to the drawings and particularly to FIGS. 1 and 2, one form of the apparatus of the invention is there shown and generally designated by the numeral 14. The apparatus here comprises a hollow housing 16 having a front panel 18 upon which a display button 20 is mounted. Front panel 18 also carries a start button 22 and a reset button 24. Disposed within the hollow housing 16—See FIG. 2—is a conventional air compressor 26 which draws air from atmosphere, compresses it and introduces the compressed air into an air tank 28 via a conduit 27.

Interconnected with air tank 28 is an elongated fluid (liquid or gas) flow conduit that comprises a first segment 30 that has a proximal end 32 and a distal end 34. Proximal end 32 is connected to air tank 28 while distal end 34 communicates with the first portion, or inflow branch 35a of an interface means, shown here as a disposable external tubing assembly 35. The second portion, or main trunk 35b of tubular assembly 35 communicates with the body cavity “B” that is to be pressurized. Disposed intermediate proximal end 32 and distal end 34 of the first segment 30 of the fluid (liquid or gas) flow conduit is a pressure gauge 36, a pressure regulator 38, a flow regulator 40 and a safety valve 42. Pressure gauge 36 is used to verify that there is sufficient pressure in conduit segment 30 to initiate the inflow cycle of one form of the method of the invention. The pressure regulator 38 ensures uniformity of pressure during the conduct of the method of the invention and the flow regulator 40 ensures uniformity of air flow through the segment 30. The safety valve 42, which communicates with atmosphere, remains closed during the operational sequences of the method unless the pressure in the system exceeds a predetermined maximum level in which case the safety valve automatically opens to vent the system to atmosphere. Also disposed intermediate the proximal and distal ends of segment 30 is an inflow valve 44 that remains in a closed position while the apparatus is in a standby mode.

As illustrated in FIG. 2 of the drawings, the third portion, or outflow branch 35c of the external tubing assembly 35 communicates with a second segment 46 of the fluid (liquid or gas) flow conduit. This second segment has a proximal end 46a and a distal end 46b that is interconnected with a disposable discharge bag 48. Disposed between the proximal and distal ends of second segment 46 are an outflow valve 50 and an outflow regulator 52 for regulating fluid (liquid or gas) flow through segment 46. Communicating with second segment 46, proximate its proximal end 46a, is the important sensor means of the invention, shown here as a pressure sensor 54. Pressure sensor 54 senses the pressure within second segment 46, generates an appropriate pressure signal and transmits the pressure signal to the control means, or microprocessor, of the invention which is housed within housing 16.

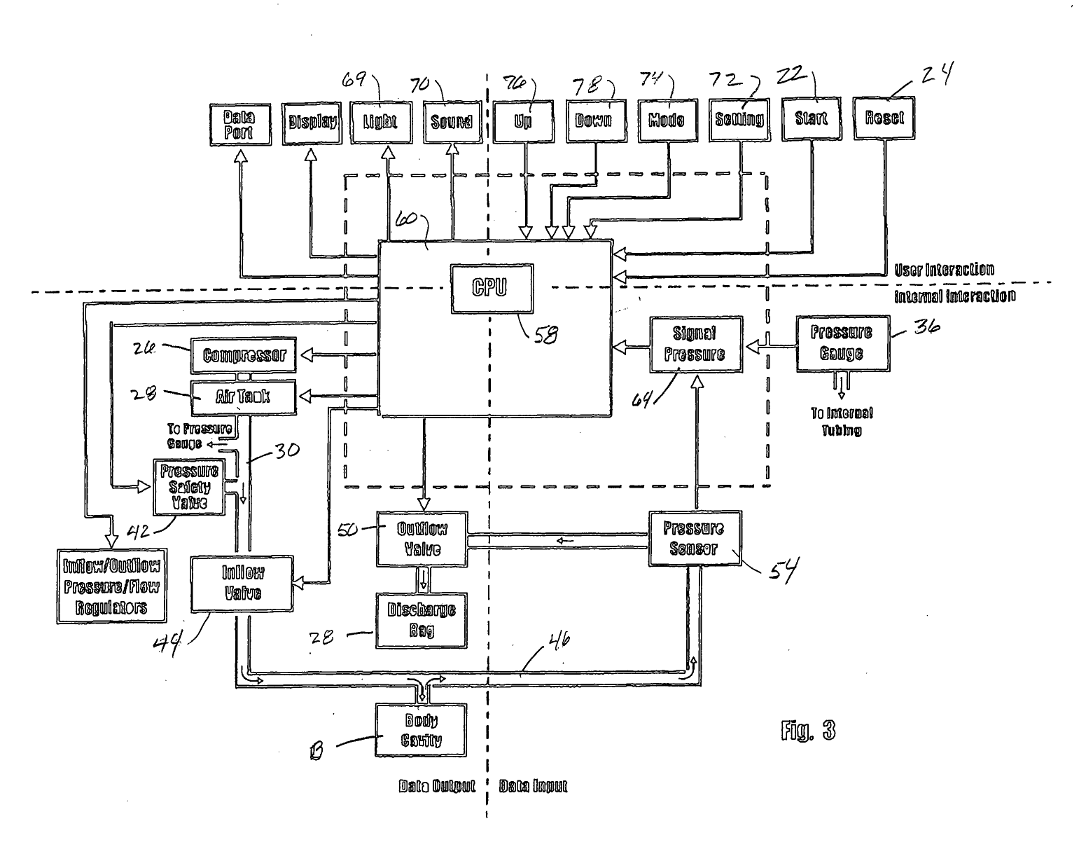

Turning also to FIG. 3 of the drawings, it can be seen that the control means of the present form of the invention comprises the central processing unit, CPU, 58 of a conventional microprocessor 60 that is interconnected with, and controls the operation of, many of the operating components that make up the apparatus of the invention.

In the conduct of one form of the method of the invention, the user first activates the apparatus by manipulating the main switch 62 (FIG. 1). Upon activation of the device, the control means, or CPU 58, which has been appropriately programmed in a manner well known to those skilled in the art, will cause the inflow valve 44 to open and will energize the compressor 26 so as to cause—See FIGS. 2 and 3—the controlled pressurization of air tank 28 and the segment 30. When the pressure in segment 30 reaches a predetermined level as determined by the pressure gauge 36, the CPU will receive an appropriate signal from the pressure gauge 36 via the signal pressure indicator 64. At this point, the CPU will cause the illumination of an illuminable green, ready light 69, which is mounted on panel 18 of housing 16, and will also cause an audio signal to be emitted by the audio signaling device 70 carried by housing 16. Upon receiving these signals the user will set the appropriate operating parameters of the method that is to be conducted. In the present form of the invention this is accomplished through use of the setting 72, the mode 74, the up 76 and the down 78 features of the apparatus (FIG. 3). After the appropriate operating parameters have been set, the user pushes the start button 22 causing the CPU to commence the testing cycle by first opening the inflow valve 44 thereby permitting the controlled pressurization of the body cavity “B”. In accordance with this method of the invention, the pressurization of the body cavity will be constantly monitored by the sensor means or pressure sensor 54. When the pressure reaches a preset level, the CPU will cause the inflow valve 44 to close, the compressor to be deenergized and the outflow valve 50 to open. Opening of the outflow valve 50 signals the commencement of the outflow, or unload, phase of the method of the invention.

At the commencement of this important outflow phase, the body cavity “B” will recoil in a manner to exert a rebound pressure that will drive the air from the body cavity, through conduit 46 and into the disposable collection bag 48 via the flow regulator 52. The end of the outflow cycle occurs when the pressure within the system drops below a predetermined level. At this time the outflow valve 50 will be automatically closed by the CPU. It is to be understood that, if during the testing process, the pressure within segment 30 exceeds a predetermined level as determined by the pressure gauge 36 and the sensor 54, the CPU will automatically deenergize the compressor 26 and will cause the safety valve 42 to automatically open.

If the user wishes to repeat the test, the reset button 24 (FIG. 1) is pushed. This will cause the opening of the outflow valve 50 for a predetermined, short period of time in order to decompress the external tubing assembly 35 and the body cavity space “B”. The CPU will then close the outflow valve 50, and close the inflow valve 44 (FIG. 3) and will energize the compressor 26. As before, when the pressure within segment 30 reaches a predetermined level the green ready light will be illuminated and the testing method can be repeated in the manner previously described.

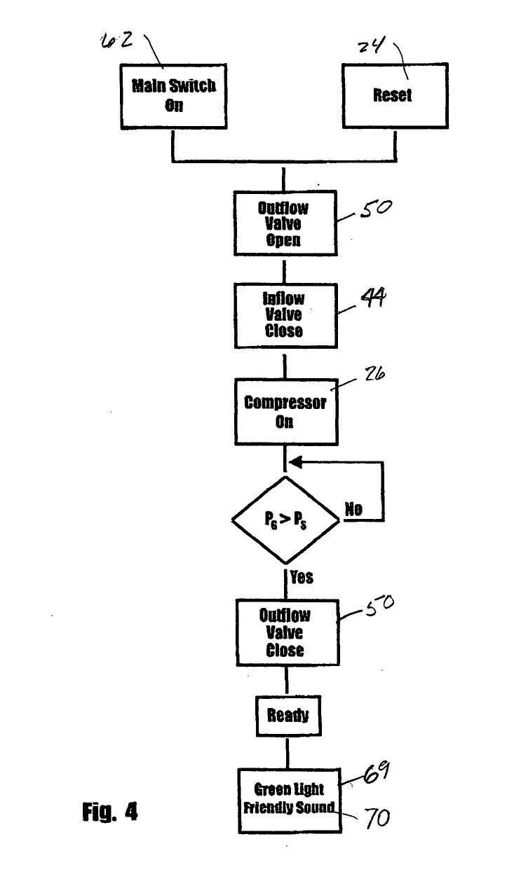

Turning next to FIG. 4 of the drawings, the priming method of the invention is there illustrated. This priming step can be accomplished in one of two ways, either by pressing the reset button 24 or by operating the main switch 62 (FIG. 1). In either case, this causes outflow valve 50 to open, the inflow valve 44 to close and the compressor 26 to be energized. The pressure within segment 30 is then determined using pressure gauge 36, and this pressure is compared with the preset starting threshold pressure. This step is repeated until the pressure within the segment 30 exceeds the desired starting threshold pressure at which point the microprocessor closes the outflow valve 50, causes the green light 69 to illuminate and causes an audio signal to be generated 70.

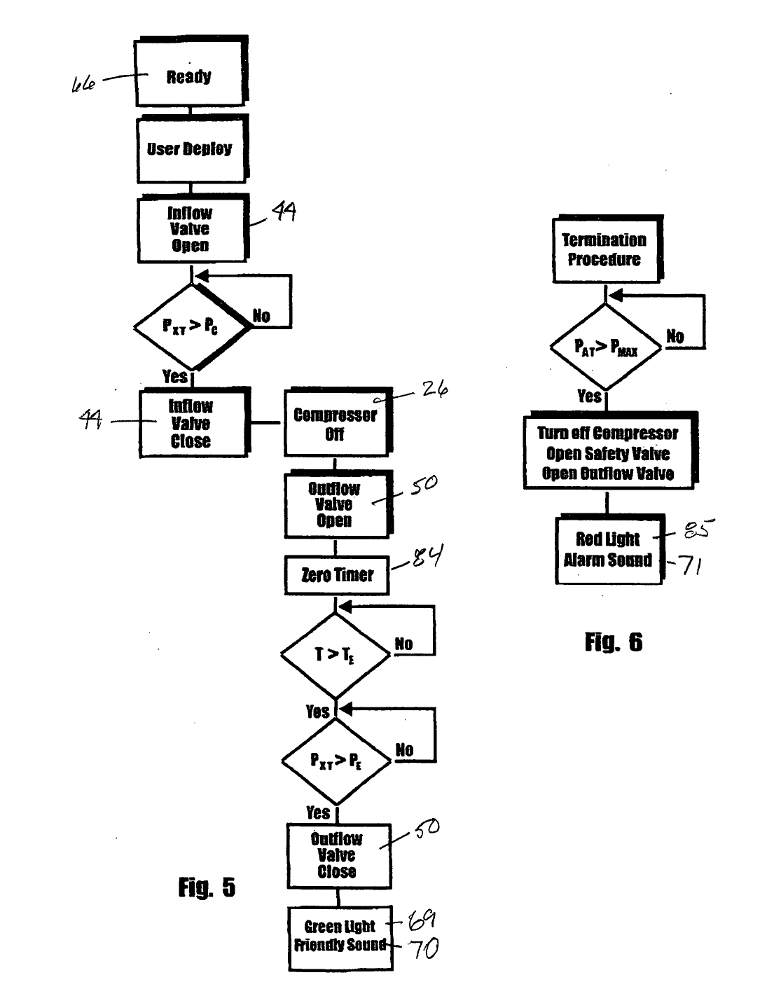

Referring next to FIG. 5 of the drawings, the method of the present invention is further illustrated there. Following appropriate programming of the microprocessor unit of the invention and after the apparatus has been interconnected with a source of electrical power “S” (FIG. 1), the ready button 66 is pressed to start the testing procedure. As indicated in FIG. 5, this causes the inflow valve 44 to open. At this point the pressure sensor 34 monitors the pressure P_{XT} in the external tubing assembly 35 and compares this pressure to a preset critical pressure. At such time that the pressure P_{XT} exceeds the predetermined critical pressure P_{C}, the CPU closes the inflow valve 44, deenergizes the compressor 26 and opens the outflow valve 50. At this time the apparatus, or Zero, timer 84, which is set at zero, is started. Once the timer reaches a predetermined elapsed time as, for example, 10 seconds, the P_{XT} is monitored using sensor means until it reaches a level less than a predetermined ending pressure P_{E}. When this pressure is reached, the cycle is finished, the green light 69 is energized and an audio signal is generated 70.

Turning to FIG. 6 of the drawings, the automatic termination feature of the method of the invention is there illustrated. This feature of the invention, which is preprogrammed in the microprocessor, is triggered when the pressure P_{AT} in the conduit 30, as measured by gauge 36 or sensor 54, exceeds a predetermined pressure P_{MAX}. When this happens, the safety valve is opened, the compressor 26 is automatically deenergized, the outflow valve 50 is automatically opened, a red signal light 85 is illuminated and an audio alarm is sounded 71.

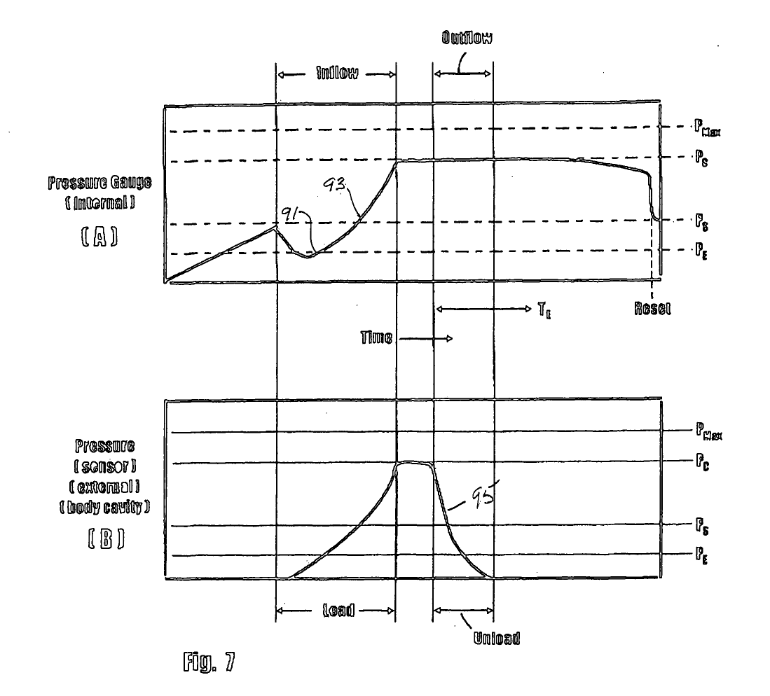

FIGS. 7A and B graphically depict pressure changes in the system as a function of time during the conduct of the diagnostic method of the invention. More particularly, FIG. 7A depicts pressure changes in segment 30 of the internal system as measured by pressure gauge 36. Similarly, FIG. 7B depicts pressure changes in the external system, or disposable assembly 35 during the loading and unloading cycles of the diagnostic method of the invention. As illustrated in FIG. 7A, as the air is introduced into the body cavity “B” at the starting pressure P_{S}, which exceeds the external pressure P_{E}, the pressure first decreases and then progressively increases as a function of time. As shown by segment 91 of FIG. 7A, the initial pressure change is relatively slow. However, as the body cavity exerts resistance to the inward flow of air, the pressure increases more rapidly as illustrated by segment 93 of FIG. 7A. When the pressure reaches a critical level P_{C}, the pressure curve flattens indicating the completion of the inflow study. The various diagnostic parameters that can be extracted from the graphical representation include but are not limited to the initial slope as depicted by segment 91, the maximum slope (93), the time to reach maximum slope, etc. Analysis of these parameters, which reveal the elastic properties of the body cavity, can be used to draw conclusions as to the presence or absence of disease in the organ that contains the cavity.

Referring to FIG. 7B, once the peak pressure P_{C} is reached the compressor is deenergized by the CPU and the outflow valve 50 is opened. This permits the air under pressure to flow from the body cavity “B” toward the discharge bag 48. As indicated by the curved segment 95 there is initially a steep decline in pressure followed by a slower decline in pressure. The parameters that can be extracted from the graphical representation include the initial slope of the curve, the peak slope P_{S} of the curve and the time to reach a 50% decline in pressure. These parameters can also be used to draw conclusions as to the condition of the organ that contains the cavity.

It is to be understood that many different types of loading and unloading studies can be performed using the apparatus of the invention to extract a variety of biomechanical parameters and functional motility characteristics of the organ under study. Furthermore, it is to be appreciated that, while the Figure drawings illustrate studies of pressure as a function of time, studies can also be undertaken involving data collection and plotting of fluid (liquid or gas) flow as a function of time, rather than pressure change as a function of time, along with other fluid (liquid or gas) mechanics correlations.

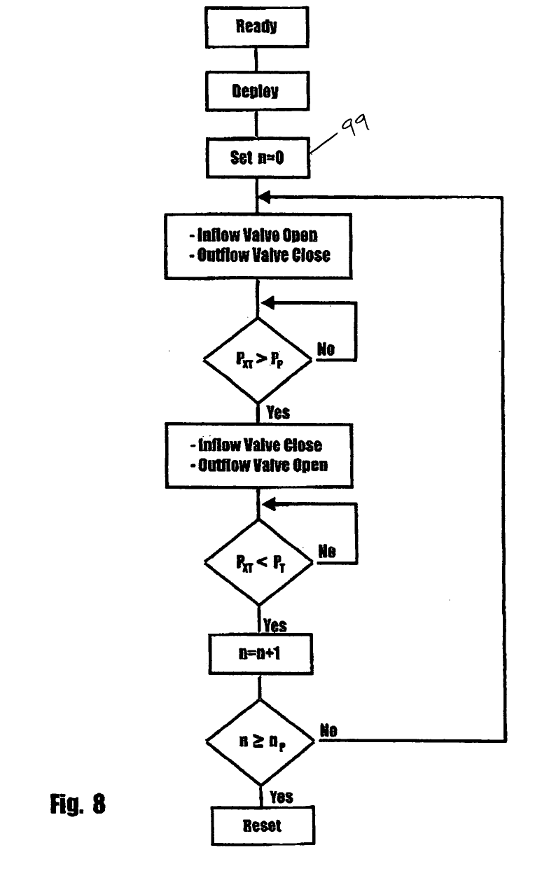

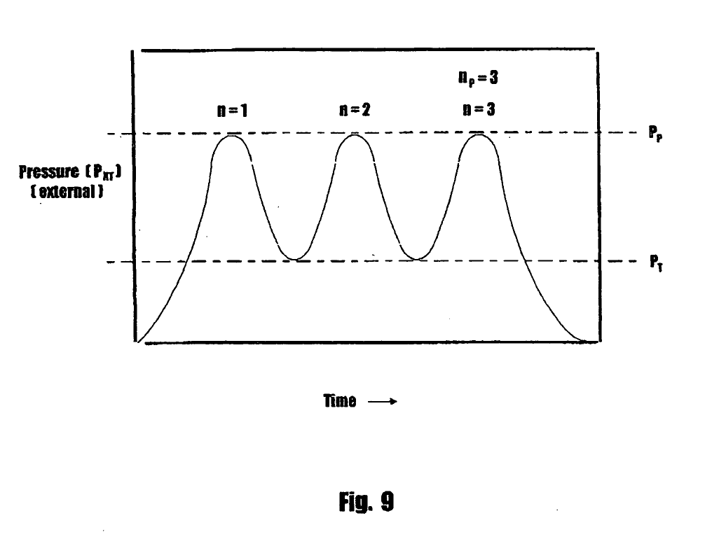

Turning to FIGS. 8 and 9 of the drawings, the various steps in an alternate form of the method of the invention for purposes of rehabilitation are there illustrated. More particularly, as previously mentioned, the apparatus can be used to train body cavities with motility dysfunction to function more normally. By way of example, this can be achieved by repeatedly inflating and deflating the body cavity. This technique can also be used to determine the strengths and weaknesses of a particular body cavity and can be customized for each individual patient. As indicated in FIG. 8, when the apparatus is in the “ready” status, the “modes” and “settings” buttons (74, 72 of FIG. 1) can be used to place the apparatus in the rehabilitation mode at the appropriate setting. When this is done the cycle counter 99 sets itself to n=0. The first cycle then begins with the microprocessor opening the inflow valve 44 and closing the outflow valve 50. This will permit controlled pressurization of the body cavity. The pressurization of the body cavity is monitored by the sensor 54 and appropriate pressure signals are transmitted to the CPU. The CPU compares the pressure P_{XT} in the external tubing system 35 with a predetermined peak pressure P_{P} (FIG. 9). At such time as the external pressure exceeds the peak pressure, the CPU automatically closes the inflow valve 44 and opens the outflow valve 50. This permits fluid (liquid or gas) outflow from the body cavity in a manner to cause a decrease in the pressure. When the pressure decreases below a predetermined pressure P_{T}, the counter 99 records the completed cycle as n=n+1. If the number of cycles is below the planned number of cycles nₚ, i.e., n<nₚ, another cycle is automatically commenced. If the number of cycles reaches or exceeds the planned number of cycles, the system will automatically go into the reset mode (FIG. 8).

It is to be understood that the methods of the invention for rehabilitation can be customized for a particular patient with the pressure levels, the number of cycles to be undertaken and a variety of modes and settings can be specially selected for that particular patient. The settings are extracted from data obtained during the diagnostic phase of the invention when used on that patient undergoing the therapeutic intervention.

I Claim:

1. An apparatus for controllably introducing fluids into a body cavity comprising:
(a) a fluid flow conduit;
(b) interface means for interconnecting said fluid flow conduit with the body cavity;
(c) pressurization means operably associated with said fluid flow conduit for introducing fluid under pressure into said fluid flow conduit;
(d) sensor means operably interconnected with said fluid flow conduit for sensing fluid pressure within said fluid flow conduit;
(e) valve means for controlling fluid flow through said fluid flow conduit;
and
(f) control means operably associated with said sensor means and said valve means for operating said valve means.

2. The apparatus as defined in claim 1 in which said interface means comprises a tubing assembly removably interconnected with said fluid flow conduit.

3. The apparatus as defined in claim 1 in which said control means comprises a microprocessor operably associated with said sensor means and said valve means for receiving signals generated by said sensor means and for operating said valve means.

4. The apparatus as defined in claim 1, further including a pressure gauge interconnected with said fluid flow conduit for measuring fluid pressure within said fluid flow conduit;

5. The apparatus as defined in claim 1 in which said pressurization means comprises a compressor connected to said fluid flow conduit.

6. The apparatus as defined in claim 1, further including an air tank disposed intermediate said pressurization means and said fluid flow conduit.

7. The apparatus as defined in claim 1, further including a safety valve interconnected with said fluid flow conduit for venting fluids contained within said fluid flow conduit to atmosphere.

8. The apparatus as defined in claim 1 in which said fluid flow conduit comprises a first segment having a proximal end and a distal end and a second segment having a proximal end and a distal end.

9. The apparatus as defined in claim 8 in which said interface means comprises a tubular assembly having a first portion interconnected with said distal end of said first segment, a second portion in communication with the body cavity and a third portion interconnected with said proximal end of said second segment.

10. The apparatus as defined in claim 9, further including a disposable bag interconnected with said third portion of said interface means.

11. An apparatus for controllably introducing fluids into a body cavity comprising:
(a) a fluid flow conduit;
(b) interface means for interconnecting said fluid flow conduit with the body cavity, said interface means comprising a tubing assembly removably interconnected with said fluid flow conduit;
(c) pressurization means operably associated with said fluid flow conduit for introducing fluid under pressure into said fluid flow conduit;
(d) a pressure gauge interconnected with said fluid flow conduit for measuring fluid pressure within said fluid flow conduit;
(e) sensor means operably interconnected with said fluid flow conduit for sensing fluid pressure within said fluid flow conduit and for generating signals corresponding thereto;
(f) valve means for controlling fluid flow through said fluid flow conduit; and
(g) control means operably associated with said sensor means and said valve means for receiving said signals from said sensor means and for operating said valve means, said control means comprises a microprocessor operably associated with said sensor means and said valve means.

12. The apparatus as defined in claim 11 in which said pressurization means comprises a compressor connected to said fluid flow conduit.

13. The apparatus as defined in claim 11, further including in an air tank disposed intermediate said pressurization means and said fluid flow conduit.

14. The apparatus as defined in claim 11, further including a safety valve interconnected with said fluid flow conduit for venting fluids contained within said fluid flow conduit to atmosphere.

15. The apparatus as defined in claim 11 further including a regulator connected to said fluid flow conduit for regulating fluid flow therethrough.

16. The apparatus as defined in claim 11 in which said fluid flow conduit comprises a first segment having a proximal end, a distal end and a second segment having a proximal end and a distal end.

17. The apparatus as defined in claim 16 in which said tubular assembly includes a first portion interconnected with said distal end of said first segment, a second portion in communication with the body cavity and a third portion interconnected with said proximal end of said second segment.

18. The apparatus as defined in claim 17, further including a disposable bag interconnected with said third portion of said interface means.

19. A method for making dynamic biomechanical measurements of the body cavity using an apparatus for controllably introducing fluids into the body cavity, said apparatus including a fluid flow conduit interconnected with the body cavity, pressurization means operably associated with the fluid flow conduit for introducing fluid under pressure into the fluid flow conduit and sensor means operably interconnected with the fluid flow conduit for sensing fluid pressure within the fluid flow conduit, said method comprising the steps of using the pressurization means and the fluid flow conduit to controllably pressurize the body cavity and using the sensor means to continuously sense, as a function of time, the pressure within the body cavity during the conduct of the pressurization step.

20. The method as defined in claim 19 including a further step of depressurizing the body cavity and using the sensor means to continuously sense, as a function of time, the pressure within the body cavity during the conduct of the depressurization step.

21. A method for rehabilitative treatment of a body cavity using an apparatus for controllably introducing fluids into the body cavity, said apparatus including a fluid flow conduit interconnected with the body cavity, pressurization means operably associated with the fluid flow conduit for introducing fluid under pressure into the fluid flow conduit and sensor means operably interconnected with the fluid flow conduit for sensing fluid pressure within the fluid flow conduit, said method comprising the steps of using the pressurization means and the fluid flow conduit to controllably pressurize the body cavity to a predetermined pressure to controllably expand the body cavity, using the sensor means to measure the pressure within the body cavity to ascertain when the predetermined pressure has been reached and, upon reaching the predetermined pressure, depressurizing the body cavity.

22. The method as defined in claim 21 including a further step of, following the pressurizing of the body cavity, using the pressurization means and the fluid flow conduit to controllably repressurize the body cavity to a predetermined pressure to controllably reexpand the body cavity.
